(19) United States
(12) Patent Application Publication
(10) Pub. No.: US 2006/0069157 A1
(43) Pub. Date: Mar. 30, 2006

(54) Method of ameliorating or abrogating the effects of a neurodegenerative disorder, such as Huntington's disease, by sodium butyrate chemotherapy

(75) Inventor: Robert J. Ferrante, Canton, MA (US)

Correspondence Address: DINESH AGARWAL, P.C., 5350 SHAWNEE ROAD, SUITE 330, ALEXANDRIA, VA, 22312, US

(73) Assignee: U.S. DEPARTMENT OF VETERANS AFFAIRS

(21) Appl. No.: 11/058,297

(22) Filed: Feb. 16, 2005

Related U.S. Application Data
(60) Provisional application No. 60/545,532, filed on Feb. 19, 2004.

(51) Int. Cl. A61K 31/19

(52) U.S. Cl. 514/557

(57) ABSTRACT

A method of ameliorating or abrogating the effects of a neurodegenerative disorder, such as Huntington's disease, includes administering a histone deacetylase (HDAC) inhibitor in a subject in need thereof. The HDAC inhibitor includes sodium butyrate.

### CROSS-REFERENCE TO RELATED APPLICATIONS

The present application claims priority on prior U.S. Provisional Application Ser. No. 60/545,532, filed Feb. 19, 2004, which is hereby incorporated herein in its entirety by reference.

### STATEMENT REGARDING FEDERALLY SPONSORED RESEARCH OR DEVELOPMENT

The work leading to the present invention was supported by one or more grants from the U.S. Government, including NIH Grant(s): NINDS, NS 045242. The U.S. Government therefore has certain rights in the invention.

### BACKGROUND AND DESCRIPTION OF THE INVENTION

Huntington's disease (HD) is a progressive and fatal neurological disorder that is caused by an expanded CAG repeat in a gene coding for a protein of unknown function, huntingtin. There are no current drug therapies proven to help ameliorate or abrogate the disease process in HD. Although the exact cause of the selective neuronal death in HD remains unknown, it has been postulated that aberrant protein-protein interactions, including aggregation of the mutant huntingtin protein, may be toxic to neurons and lead to oxidative stress, mitochondrial dysfunction, apoptosis, energy metabolism defects, and excitotoxicity (Beal, 2000; Friedlander 2003). The seminal event in this cascade may be transcriptional dysregulation initiated by direct binding of the mutant huntingtin protein or cleaved products to a number of transcription factors, disrupting the normal pattern of gene transcription and resulting in functional and degenerative changes (Cha, 2000, McCampbell et al., 2000; Steffan et al., 2000 and 2001; Sugars and Rubinsztein, 2003). Changes in gene expression occur very early in polyglutamine transgene models of disease (Cha, 2000). Gene expression profiling has shown that mutant huntingtin protein selectively affects the pattern of gene expression (Luthi-Carter et al., 2000). These mRNA changes may affect neuronal functioning. A key strategy in the treatment of HD may, therefore, be to modulate transcription that leads to altered signaling-cascades, underlying the HD pathology.

Transcription is regulated by complex interactions between many proteins, among them transcription factors and histones that ultimately affect the actions of DNA polymerase II on individual genes. Many of these interactions, in turn, are regulated by covalent modifications such as acetylation, methylation, and phosphorylation. Histone acetylation modulates a subset of genes and is determined by interplay between histone acetyltransferases and histone deacetylases.

Mutant huntingtin can bind to histone acetyltransferase domains and reduce this activity, resulting in a reduction in histone acetylation and in repressed gene transcription (Steffan et al., 2000). Drugs that prevent the resulting histone deacetylation can help restore transcription in the presence of mutant huntingtin (McCampbell et al., 2001; Steffan et al., 2001). These compounds, known as histone deacetylase (HDAC) inhibitors, affect histones as well as transcription factors that are regulated by acetylation. HDAC inhibitors promote transcriptional activation by relaxing the DNA conformation. They are selective in that only 2-5% of genes are affected (Van Lint et al., 1996). Because HDAC inhibitors induce growth arrest in cell proliferation models, HDAC inhibitors are currently under development as anti-cancer drugs (Richon et al., 2000; Bulter et al., 2000; Vigushin and Coombes, 2002). The most widely studied compounds have been, sodium butyrate, phenylbutyrate, trichostatin A, and suberoylanilide hydroxamic acid (SAHA). The butyrates, however, have been the best clinically-studied compounds and are known to readily reach the brain (Egorin et al., 1999).

Both SAHA and sodium butyrate slow photoreceptor neuron degeneration and ameliorate lethality in a Drosophila model of HD (Steffan 2001). Using cyclodextrin as a carrier, SAHA has also been reported to increase histone levels and improve motor performance in transgenic HD mice (Hockly et al., 2003).

We, therefore, investigated the effects of sodium butyrate on the clinical and neuropathological phenotype of the R6/2 transgenic mouse model of HD, on histone and Sp1 acetylation, and gene expression in R6/2 brain.

In my co-pending Application Ser. No. 60/617,643, filed Oct. 13, 2004, entitled METHOD OF AMELIORATING OR ABROGATING THE EFFECTS OF A NEURODEGENERATIVE DISORDER, SUCH AS HUNTINGTON'S DISEASE, BY USING COENZYME Q₁₀, (hereby incorporated herein in its entirety by reference), the effects of coenzyme Q₁₀ on HD pathogenesis were shown. The present invention is directed to the effects of intraperitoneal administration of histone deacetylase (HDAC) inhibitor, such as sodium butyrate, on HD pathogenesis.

### OBJECTS AND SUMMARY OF THE INVENTION

The principal object of the present invention is to provide a method of ameliorating or abrogating the effects a neurodegenerative disorder, such as Huntington's disease, by using a HDAC inhibitor, such as a butyrate, and specifically sodium butyrate.

An object of the present invention is to provide a method of ameliorating or abrogating the effects of a neurodegenerative disorder, such as Huntington's disease, by high dose administration of a HDAC inhibitor, such as a butyrate, and specifically sodium butyrate.

Another object of the present invention is to increase survival, neuroprotection, improve motor performance, improve gross brain weight and atrophy, and striatal neuron atrophy, and/or prevent body weight loss by administering a HDAC inhibitor, such as a butyrate, and specifically sodium butyrate, in a subject.

An additional object of the present invention is to demonstrate therapeutic effects of administration of a HDAC inhibitor, such as a butyrate, and specifically sodium butyrate, in the R6/2 mice with Huntington's disease.

In summary, the main object of the present invention is to provide a method of ameliorating or abrogating the effects of a neurodegenerative disorder, such as Huntington's disease, by using a HDAC inhibitor, such as a butyrate, and specifically sodium butyrate.

One of the above objects is met, in part, by the present invention which in one aspect includes ameliorating or abrogating the effects of a neurodegenerative disorder in a subject by administering a HDAC inhibiting agent in a subject in need thereof.

Another aspect of the present invention includes preventing neuronal death in a subject having Huntington's disease by administering a HDAC inhibiting agent in a subject in need thereof.

Another aspect of the present invention includes treating a subject having Huntington's disease by administering a HDAC inhibiting agent in a subject in need thereof.

Another aspect of the present invention includes protecting neural cells in a subject having a neurological disorder by increasing histone acetylation by administering a butyrate in a subject in need thereof.

Another aspect of the present invention includes improving motor performance, gross brain weight and atrophy, and striatal neural atrophy, and/or survival in a subject by administering a HDAC inhibiting agent in a subject in need thereof.

### BRIEF DESCRIPTION OF THE DRAWINGS

One of the above and other objects, novel features and advantages of the present invention will become apparent from the following detailed description of the preferred embodiment(s) of the invention, as illustrated in the drawings, in which:

FIG. 1. Survival, body weight, and motor performance analysis in sodium butyrate-treated R6/2 mice. Cohorts of R6/2 mice (n=20) were treated from day 21 with 100, 200, 400, 600, 1,200, 5,000, and 10,000 mg/kg/d sodium butyrate intraperitoneal injection. Kaplan-Meier probability of survival analyses of sodium butyrate treatments in R6/2 mice and PBS-treated R6/2 mice are presented in FIG. 1A. Survival was significantly extended in all sodium butyrate dosing paradigms except the 100 mg/kg/d dose, with the greatest increase at the 1,200 mg/kg/d dose. The data for the 5,000 and 10,000 mg/kg/d doses resulted in marked rapidly-coursing morbidity and mortality at onset of treatment and are not included. Effects of intraperitoneal sodium butyrate treatment on body weight in R6/2 HD transgenic mice is shown in FIG. 1B. Significant reduction in body weight loss was observed only after 12 weeks of age. Effects of intraperitoneal sodium butyrate treatment on rotarod performance (C) significantly improved motor performance in R6/2 HD transgenic mice throughout the temporal sequence of the experiment in each of the doses tested. * p<0.01, ** p<0.001.

FIG. 2. Gross brain and histopathological neuroprotection with sodium butyrate treatment. Photomicrographs of coronal serial step sections from the rostral neostriatum through the level of the anterior commissure in wild type littermate mouse (A1-4), sodium butyrate-treated (1.2 g/kg/d) R6/2 HD transgenic mouse (B1-4), and PBS-treated (C1-4) R6/2 HD transgenic mouse at 90 days. There was gross atrophy of the brain in the PBS-treated R6/2 mouse along with ventricular hypertrophy (C1-4), in comparison to wild type littermate control mouse (A1-4). In contrast, the sodium butyrate-treated R6/2 mouse brain (B1-4) showed reduced gross brain atrophy and ventricular enlargement, in comparison to the PBS-treated R6/2 mouse (C1-4). Corresponding Nissl-stained tissue sections from the dorso-medial aspect of the neostriatum in wild type littermate control (A5), sodium butyrate-treated R6/2 mouse (B5), and PBS-treated R6/2 mouse (C5). There was marked neuronal atrophy in the PBS-treated R6/2 mouse, with significantly less neuronal atrophy (p<0.01) in the sodium butyrate-treated R6/2 mouse, in comparison PBS-treated R6/2 mouse. The histogram shows mean and standard deviation of somal areas of striatal neurons quantitated in each group of mice (n=10) (see Methods). Scale bar in A equals 2 mm and is the same for B and C. Scale bar in A5 equals 100 _m and is the same for B5 and C5.

FIG. 3. Huntingtin and ubiquitin immunoreactivity in sodium butyrate-treated R6/2 mice. Huntingtin immunostained tissue sections from the neostriatum of PBS-treated R6/2 transgenic mouse (A) and sodium butyrate-treated R6/2 HD transgenic mouse euthanized at 90 days of age (B). Sodium butyrate (1.2 g/kg/d) and PBS treatments were started at 21 days. There were no significant differences (p<0.27) in the number and size of huntingtin aggregates and inclusions between treated and untreated mice. Similarly, no differences were observed in ubiquitin-positive inclusions between PBS-treated and sodium butyrate-treated R6/2 mice (C and D, respectively). Scale bar in B equals 100 _m and is the same for all photomicrographs.

FIG. 4. Western blot of acetylated histone 3 and histone 4 in sodium butyrate-treated R6/2 mice. R6/2 mice were treated with 1.2 g/kg/d sodium butyrate for two weeks starting at 42 days and euthanized at 56 days. Hypoacetylation of H3 and H4 immunoreactivities were present in R6/2 mice, in comparison to wild type littermate control mice (WT). Sodium butyrate-treated R6/2 mice showed a marked increase in acetylated H3 and H4 activity. Protein levels were determined using Coomassie Protein Assay.

FIG. 5. Striatal tissue immunohistochemistry of acetylated histone 4 in sodium butyrate-treated R6/2 mice. At 90 days of age, robust acetylated histone 4 immunohistochemistry was present in wild type littermate control striatal tissue specimens (A), with hypoacetylation in the R6/2 mice (B). Sodium butyrate treatment (1.2 g/kg/d) increased acetylation of histone 4 in R6/2 mice (C). Bar in A equals 100 _m and is the same for B and C.

FIG. 6. The histone deacetylase inhibitor, sodium butyrate, enhances Sp1 acetylation in vivo. Cohorts of R6/2 mice (n=6) were treated daily with sodium butyrate (1.2 g/kg) or PBS intraperitoneal injections for two weeks. Sp1 acetylation levels from homogenized brains of PBS- and sodium butyrate-treated R6/2 were determined by immunoprecipitation, using an Sp1 antibody followed by immunoblotting using acetyl lysine antibody (Ac-Sp1) or Sp1 antibody alone (Sp1). Sp1 acetylation was increased in sodium butyrate-treated R6/2 mice. Note that levels of Sp1 did not change with sodium butyrate treatment.

FIG. 7. Sodium butyrate neuroprotection from 3-nitropropionic (3-NP) acid toxicity in R6/2 mice. Groups of R6/2 mice (n=10) were treated for two weeks with 1.2 g/kg/d sodium butyrate or PBS starting at 42 days. 3-NP was administered at the beginning of the second week for 4.5 days along with PBS and sodium butyrate treatments. Sodium butyrate treatment prevented 3-NP striatal-induced damage in R6/2 mice (A), as compared to PBS-treated R6/2 mice (B). Histopathological evaluation of 3-NP induced striatal lesions showed bilateral striatal lesions, areas of pallor, in PBS-treated R6/2 mice (B). Bar equals 2 mm.

FIG. 8. Huntingtin expression in sodium butyrate-treated R6/2 mice. Transgene expression was determined in whole brain samples by Western blot analysis of sodium butyrate-treated (1.2 g/kg/d) R6/2 mice, PBS-treated R6/2 mice, and wild type mice. No differences in huntingtin expression-levels in sodium butyrate-treated R6/2 mice were observed, as compared to PBS-treated mice. Parallel blots probed against (-tubulin were run to normalize for gel loading.

FIG. 9. MKP-1 is increased by sodium butyrate treatment. Northern blot analysis of 2 Êg total RNA from brain samples of sodium butyrate-treated (1.2 g/kg/d) and untreated R6/2 mice (n=4 in each group) confirmed that sodium butyrate-treatment increases the expression of MKP-1 mRNA, in comparison to both untreated R6/2 mice and wild type littermate control mice. .-actin hybridization signal on the same blot (shown) was used as a loading control. Quantitative and statistical data are presented in Table 2.

### DETAILED DESCRIPTION OF THE PREFERRED EMBODIMENT(S) OF THE INVENTION

The present invention is based, in part, on the discovery that compounds, known as histone deacetylase (HDAC) inhibitors, affect histones as well as transcription factors that are regulated by acetylation.

Methods

Animals: The Bedford VA Medical Center laboratories have maintained a stable colony of R6/2 HD mice for over 6 years, with founders originating from Jackson Laboratories (Bar Harbor, Me., USA). Male transgenic HD mice of the R6/2 strain were bred with females from their background strain (B6CBAFI/J). Offspring were genotyped using a PCR assay on tail DNA. We have standardized criteria to ensure homogeneity of the cohorts within the testing groups (Dedeoglu et al., 2002). Mice were randomized from 28 litters all within 3 days from the same ‘f’ generation. Body weights were taken at 20 days and mice were equally distributed according to weight within each cohort. Mice under 7 g at 20 days were excluded from the experiments. The animals were housed 5/cage under standard conditions with free access to water and food. The mice were handled under the same conditions by one investigator. Since we have not observed gender differences in survival in the R6/2 transgenic HD mouse model (Ferrante et al., 2000), female mice were used in the experimental paradigms. These experiments were carried out in accordance with the NIH Guide for the Care and Use of Laboratory Animals, and were approved by both the Veterans Administration and Boston University Animal Care Committees.

Intraperitoneal Dosing: Based upon previous studies (Egorin et al., 1999), a dose response study was performed, treating groups of wild type mice (n=10) and littermate R6/2 mice (n=20) with 100, 200, 400, 600, 1,200, 5,000, and 10,000 mg/kg daily intraperitoneal injection (100 Êl) of sodium butyrate (Acros Organics/Fisher Scientific) dissolved in PBS and made fresh daily. Control groups were treated with PBS injection or untreated. Approximately 240 mice were used for behavioral and survival analyses.

Clinical Assessment: Both motor performance and body weight were measured throughout the study. Training sessions were given on days 21 and 22 to acclimate the mice to the rotarod apparatus (Columbus Instruments, Columbus, Ohio). Motor performance (constant rotation at 16 rpm) was assessed weekly from 23-63 days of age and twice weekly from 63 days of age in the sodium butyrate-treated, PBS-treated R6/2 mice, and wild type littermate control mice. Three 60 sec trials were given during each session and averaged. Body weights were recorded twice weekly at the same time of day in all groups.

Survival: R6/2 mice were assessed for morbidity and mortality twice daily, mid morning and late afternoon. Motor performance and ability to feed were closely monitored and used as the basis for determining when to euthanize the mice. The criterion for euthanization was the point in time the HD mice were unable to right themselves after being placed on their back. In addition, deaths occurred overnight and were recorded the following morning. Two independent observers confirmed the criterion for euthanization (RJF and JKK).

Histone Acetylation Assay: At 42 days, groups of 10 R6/2 mice and littermate wild-type control mice were treated with daily with the most optimal dose of sodium butyrate (1.2 g/kg), as determined by survival studies, or PBS via intraperitoneal injections for two weeks. The mice were euthanized at 56 days of age and the brains rapidly frozen and stored at −80 βC. Histone acetylation was determined, using a previously reported Western analysis method (Warrel et al., 1998). Histones were isolated from sodium butyrate treated and untreated whole brains. Western blot analysis for histone acetylation was performed using acetylated histone H3 and acetylated histone H4 antibodies (Upstate Biotechnology, Lake Placid, N.Y.). Protein levels were determined via Coomassie Protein Assay (Pierce, Rockford, Ill.). Each of the sample measurements was carried out twice, with the investigator performing the assays (JKK) blind to genotype and treatment group.

Sp1 Acetylation Immunoprecipitation and Western Blot Analysis: At 42 days of age, groups (n=6) of R6/2 mice were treated with daily 1.2 g/kg sodium butyrate or PBS intraperitoneal injections for two weeks. The mice were euthanized and tissue lysates were obtained by homogenizing each brain sample with 100 mM Tris (pH 7.4) buffer containing 1% Triton-X 100, 150 mM NaCl, 1 mM sodium orthovanadate, 5 mM sodium fluoride, 3 mM PMSF, 3 mM DTT, 0.5 Êg/ml leupeptin, and 10 Êg/ml aprotinin. To monitor Sp1 acetylation in vivo, tissue lysates were precleared by the addition of 30 Êl of protein A-sepharose (50% v/v slurry) for 1 hr at 4° C. and incubated with 2 Êg of Sp1 antibody for 2-4 hr. Twenty-five Êl of protein A-sepharose was added to lysates and left for 1 hr at 40 C. All beads were collected by centrifugation and washed twice with lysis buffer and once with PBS. The samples were boiled and divided into equal aliquots before separation on SDS-PAGE. Samples were electrophoresed under reducing conditions on 8% polyacrylamide gels. Proteins were then transferred to nitrocellulose membrane (BioRad, Hercules, Calif.). Non-specific binding was inhibited by incubation in Tris buffered saline (TBST: 50 mM Tris HCl, pH 8.0, 0.9% NaCl, 0.1% Tween 20) containing 5% non-fat dried milk for 0.5 h. Primary antibodies against Sp1 (PEP2, Santa Cruz Biotechnology, Santa Cruz, Calif.) were diluted at 1:1000 in 1% milk TBST and exposed to membranes overnight at 4° C. Proteins immunoreactive to acetyl lysine were detected by using an acetyl-lysine specific antibodies (Upstate Biotechnology, Lake Placid, N.Y., 1:1000 dilution). Immunoreactive proteins were detected according to an enhanced chemiluminescent protocol (Amersham, Piscataway, N.J.). The investigators performing assays (JHL,HR) were blind to the treatment groups.

3-Nitropropionic Acid Administration: At six weeks, groups of 10 R6/2 mice were treated with daily 1.2 g/kg sodium butyrate or PBS via intraperitoneal injections for two weeks. At the start of week two, 3-nitropropionic acid (3-NP) (Sigma, St. Louis, Mo.) was dissolved in PBS (pH adjusted to 7.4), made fresh daily, and injected intraperitoneally 9 times at 12 hrs intervals (75 mg/kg) in R6/2 mice. The mice were euthanized 8-10 hrs after the last 3-NP injection, deeply anesthetized and transcardially perfused with buffered 4% paraformaldehyde and processed for histopathologic evaluation. Glycerol cryoprotected brains were frozen sectioned at 50 Êm, stained with cresyl violet, and quantitative analysis of lesion volumes was performed in serial sections as described below.

Neuropathological Evaluation of Sodium Butyrate Treatment: Beginning at 21 days, R6/2 transgenic mice and wild-type littermate control mice were treated daily with the most optimal dose of sodium butyrate (1.2 g/kg), as determined by survival studies, or PBS intraperitoneal injections. Groups of 10 animals from each treatment paradigm were deeply anesthetized and transcardially perfused with 4% buffered paraformaldehyde at 90 days of age. Forty mice were used for neuropathological analysis, as previously described (Ferrante et al., 2002). Serially cut tissue sections were stained for Nissl substance and immunostained for huntingtin (monoclonal huntingtin antibody, 1:1,000 dilution, Chemicon International Inc.) and for acetylated histone 3 and histone 4 (dilution, 1:1,000, Upstate Biotechnology, Lake Placid, N.Y.), using a previously reported conjugated secondary antibody method in murine brain tissue samples (Ferrante et al., 2002). Specificity for the antisera used in this study was examined in each immunochemical experiment to assist with the interpretation of the results. Preabsorption with excess target proteins, omission of the primary antibodies, and omission of secondary antibodies were performed to determine the amount of background generated from the detection assay.

Stereology/Quantitation: Serial-cut coronal tissue-sections beginning from the most rostral segment of the neostriatum to the level of the anterior commissure (Inter aural 5.34 mm/Bregma 1.54 mm to Inter aural 3.7 mm/Bregma −0.10 mm), were used for huntingtin aggregate analysis. Unbiased stereological counts of huntingtin-positive aggregates (>1.0 mm) were obtained from the neostriatum in 10 mice each from sodium butyrate-treated (1.2 g/kg) and PBS-treated R6/2 mice at 90 days using Neurolucida Stereo Investigator software (Microbrightfield, Colchester, Vt.). The total areas of the rostral neostriatum were defined in serial sections in which counting frames were randomly sampled. The optical dissector method was employed estimating the number of huntingtin-positive aggregates. Striatal neuron areas were analyzed by microscopic videocapture using a Windows-based image analysis system for area measurement (Optimas, Bioscan Incorporated, Edmonds, Wash.). The software automatically identifies and measures profiles. All computer identified cell profiles were manually verified as neurons and exported to Microsoft Excel. Cross-sectional areas were analyzed using Statview.

Transgene Expression: Transgene expression was determined in whole brain by Western blot analysis of groups (n=6) of sodium butyrate-treated (1.2 g/kg/d) and untreated R6/2 mice. Brain tissue was homogenized in a protease inhibitor cocktail buffer (Complete, Roche). Twenty Êg of protein (supernatant fraction) was resolved on 10% SDS-polyacrylamide gel (BioRad), transferred to PVDF membrane (BioRad), and probed with 1C2 antibody (Euromedex, France) specific for regions of expanded huntingtin, using a horseradish conjugated anti-rabbit secondary antibody, followed by chemiluminescent detection (Western Lightening™, New England Nuclear), and run with parallel blots probed against (-tubulin to normalize for gel loading.

Microarray Gene Expression Analysis: Beginning at six weeks of age, four mice were treated for 2 weeks with daily intraperitoneal injections (1.2 g/kg) of sodium butyrate. Mice were euthanized within 60 minutes of the last injection and cortical and striatal brain tissues were dissected separately and snap frozen on dry ice. In parallel, samples from four untreated R6/2 and four wild-type mice were collected in the same manner. One microarray sample was prepared from 10 Êg of total RNA from each tissue and hybridized to one U74Av2 array (Affymetrix). Four independent pair-wise comparisons were performed using MAS 5.0 software (Affymetrix) to evaluate gene expression changes between sodium butyrate-treated R6/2 and untreated R6/2 animals and between untreated R6/2 and untreated wild-type animals. Difference calls were scored, and only mRNAs that received difference calls in the same direction (increased or decreased) in at least 3 of the 4 pair-wise comparisons were included for presentation in the present study. Probability statistics for changes in the expression of these same mRNAs showed corresponding values of p<0.003 in all individual array comparisons called ‘increased’ or ‘decreased’.

Real-time PCR: Reverse transcription of 1 Êg total RNA was conducted with a SuperScript™ First-Strand Synthesis System for RT-PCR (Invitrogen) using random hexamer primers per the manufacturer's instructions. Quantitative real-time PCR studies utilized a Bio-Rad iCycler to follow the amplification of cDNA products by using SYBR_ Green PCR Master Mix (Applied Biosystems) through 50 PCR cycles (95_C for 30 s, 57_C for 1 min, 72_C for 1.5 min). Specific amplification of the target sequences was determined by melt curve analysis and DNA sequencing. Primer pair sequences were as follows: Preproenkephalin (XM_{—}131313) GTGTCCAGGCCCGAGTTC and TCTCCCGTTCCCAGTAGCTC; MKP-1 (NM_{—}013642) AAGCAGAGGCGGAGTATCAT and TAGTTCAGGGCACTGTTCGT; _-globin (NM_{—}008218) CCACCCTGCCGATTTCA and CCGCAGMGGCAGCTTAAC; _-globin (NM_{—}008220) CTTGGACCCAGCGGTACTTT and CCCAGCACAATCACGATCA; _-actin (X03672) AGGTATCCTGACCCTGAAG and GCTCATTGTAGAAGGTGTGG. Expression of the mRNAs of interest was calculated using the equation:
V=(1+E reference)(C t reference)/(1+E target) (C t target)
to correct for potential differences in RNA input and PCR primer efficiencies (Livak and Schmittgen, 2001). V=relative value of target gene normalized to reference (_-actin), E=primer efficiency, C t=threshold crossing cycle number. Differences between genotype and treatment groups were assessed using an unpaired, two-tailed Student's t-test.

Northern Blotting: Two micrograms of total RNA from brain samples of sodium butyrate-treated and untreated R6/2 and wild type mice (n=4 each) were separated on a 1.2% agarose 3% formaldehyde gel in 1× MOPS buffer, electrophoretically transferred to a nylon membrane (Genescreen Ill.) in 1× TAE, and dried in a standard utility oven at 65 βC. cDNA probes were prepared from IMAGE clones (Research Genetics, _-globin 555069, _-globin 318205, MKP-1 4235972), or cDNAs described in previous studies (enkephalin and _-actin probe sequences) (Luthi-Carter et al., 2002). Hybridizations and washes were conducted as previously described (Luthi-Carter et al. 2002). Blots were quantitated using a Molecular Dynamics Phosphorimager and its accompanying ImageQuant software. Signals for target mRNAs were expressed as ratios to _-actin signals (on the same blot), and the data was analyzed by unpaired two-tailed Student's t-test.

Statistics: The data are expressed as the mean+standard error of the mean. Statistical comparisons of rotarod, weight data, and histology data were compared by ANOVA or repeated measures ANOVA. Survival data was analyzed by the Kaplan-Meier survival curves.

Results

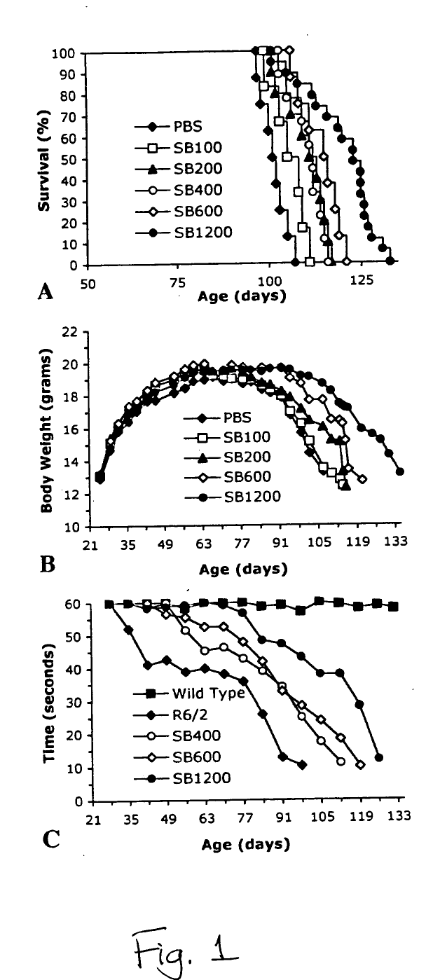

The dose response effects of intraperitoneal injection of sodium butyrate at 100, 200, 400, 600, and 1,200 mg/kg/d on survival in HD R6/2 transgenic mice are shown in FIG. 1A. Intraperitoneal administration of sodium butyrate at 1,200 mg/kg/d significantly extended survival in R6/2 mice by 20.8% (PBS-treated: 101.3 Ò 3.3 d; sodium butyrate-treated: 122.1 Ò 8.5 d; F6,120=16.28, p<0.001). Significant prolongations of survival were also observed to a lesser extent at the 200, 400, and 600 mg/kg/d doses, with no effects observed with intraperitoneal sodium butyrate treatment at 100 mg/kg/d (100 mg/kg/d sodium butyrate-treated R6/2 mice: 106.2 Ò 2.4 d, F6,120=1.81, p<0.072; 200 mg/kg/d sodium butyrate-treated R6/2 mice: 111.0 Ò 5.4 d, F6,120=6.59, p<0.01; 400 mg/kg/d sodium butyrate-treated R6/2 mice: 110.8 Ò 4.4 d, F6,120=6.23, p<0.01; 600 mg/kg/d sodium butyrate-treated R6/2 mice: 114.5 Ò 5.1 d, F6,120=9.74, p<0.01). The mice became moribund and died between 1-11 days at 5 g/kg/d sodium butyrate with sudden death occurring within 0.5-2.0 h at 10 g/kg/d sodium butyrate intraperitoneal injection after treatment was initiated at 21 days.

Intraperitoneal sodium butyrate treatment (400 mg/kg/d, 600 mg/kg/d, and 1,200 mg/kg/d) significantly improved rotarod performance throughout the entire measurement period (5-17 weeks), in contrast to PBS-treated R6/2 mice (PBS-treated R6/2 mice: 36.1 Ò 7.5 sec; 400 mg/kg/d sodium butyrate-treated R6/2 mice: 47.5 Ò 4.8 sec, 600 mg/kg/d sodium butyrate-treated R6/2 mice: 49.8 Ò 4.3 sec, 1,200 mg/kg/d sodium butyrate-treated R6/2 mice: 55.5 Ò 2.9 sec, F4,40=12.83; sodium butyrate 1,200 mg/kg/d vs PBS-treated, p<0.001). The data represent combined means from 5-14 wks (FIG. 1B). The percentile increase in rotarod performance at 90 days for the 400, 600, and 1,200 mg/kg doses was 24.0%, 27.5%, and 34.9%, respectively, in comparison to PBS-treated R6/2 mice.

Unlike other treatment regimens reported in R6/2 mice in which reduced weight-loss was observed early in the disease process (6-7 weeks) (Ferrante et al., 2000 and 2002a; Dedeoglu et al., 2003), significant weight differences were only observed after 11 weeks of treatment and continued until death (FIG. 1C). The weight curves of sodium butyrate-treated and PBS-treated mice closely paralleled one another, maintaining a plateau at 19-20 gms until death ensued within each treatment group (FIG. 1C). Significant differences in body weight occurred as an epiphenomenon of survival extension and not as a primary independent event related to sodium butyrate treatment.

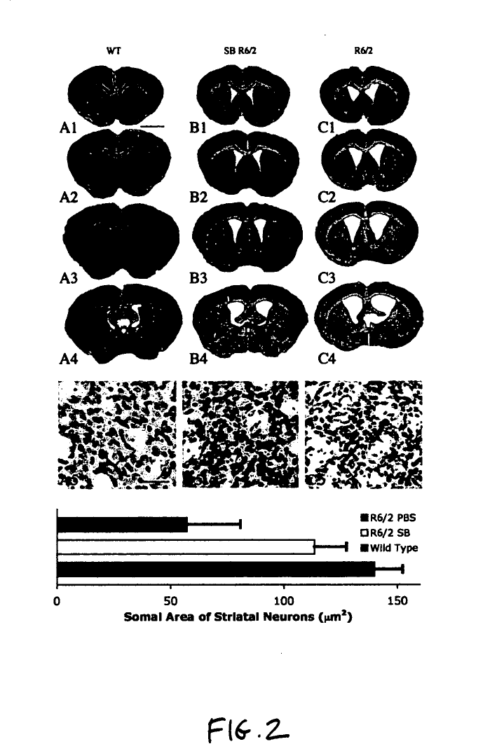

At 90 days, marked neuroprotection was observed. Similar to previous studies, there was an 18.9% reduction in brain weight in unsupplemented R6/2 mice, in comparison to wild type littermate control mice. In contrast, there was only a 5.6% brain weight loss in the R6/2 mice treated with the most efficacious dose of sodium butyrate, 1.2 g/kg/d (Wt littermate mice 447 Ò 12 mg; PBS-treated R6/2 mice: 363 Ò 24; 1.2 g/kg sodium butyrate-treated R6/2 mice: 422 Ò 17, F3,30=12.54, sodium butyrate vs PBS, p<0.001; F3,30=1.98, sodium butyrate vs Wt, p<0.53). Serial cut coronal tissue sections revealed gross brain atrophy, bilateral ventricular hypertrophy, and flattening of the medial aspect of the striatum in the PBS-treated R6/2 brains (FIG. 2). Sodium butyrate treatment (1.2 g/kg/d) ameliorated these gross neuropathological sequelae in R6/2 mice, in comparison to untreated mice at this time point. While marked striatal neuron atrophy was present in untreated R6/2 mice at 90 days, the neuroprotective effects of sodium butyrate treatment (1.2 g/kg/d) significantly reduced striatal neuron atrophy in R6/2 mice by one fold (Wt littermate control: 138.9+12.6 Êm2; sodium butyrate-treated R6/2 mice: 113.5+14.2 _m2; PBS-treated R6/2 mice: 57.1+23.7 _m2; F3,30=15.03; sodium butyrate vs PBS, p<0.01) (FIG. 2).

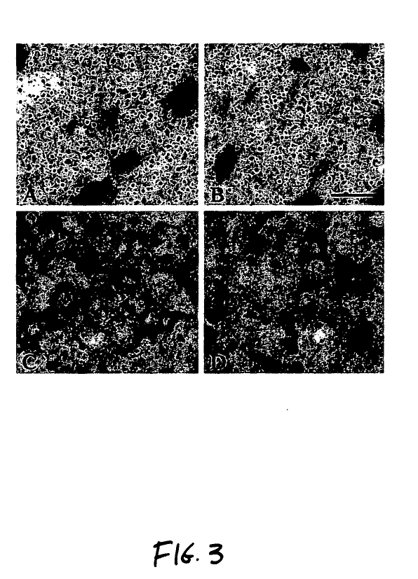

There is an early and progressive accumulation of huntingtin-immunoreactive aggregates in R6/2 mice (Ferrante et al., 2000). In contrast to most other compounds that have been efficacious in the R6/2 transgenic mice, sodium butyrate-treatment resulted in no significant reduction in huntingtin-positive striatal aggregates or ubiquitin-positive inclusions at 90 days of age, in comparison to age-matched PBS-treated R6/2 mice (Huntingtin Aggregates in sodium butyrate-treated R6/2 mice: 5.02×106+1.07; PBS-treated R6/2 mice: 5.31×106+1.21, F2,22=1.23; p<0.27) (FIG. 3). We have reported a similar dissociation between efficacy and aggregate suppression using mithramycin, an aureolic acid antibiotic that binds to G-C rich DNA sequences (Ferrante et al., 2002b). In common with sodium butyrate, mithramycin may also act to modulate transcription by displacing transcriptional activators that bind to G-C-rich regions of promoters (Miller et al., 1999; Chatergee, et al., 1999).

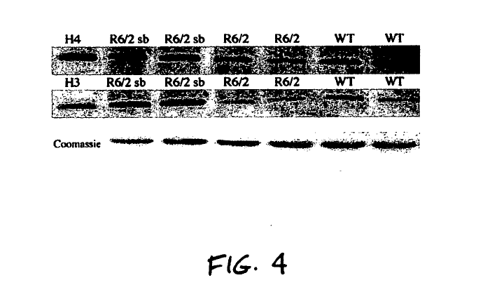

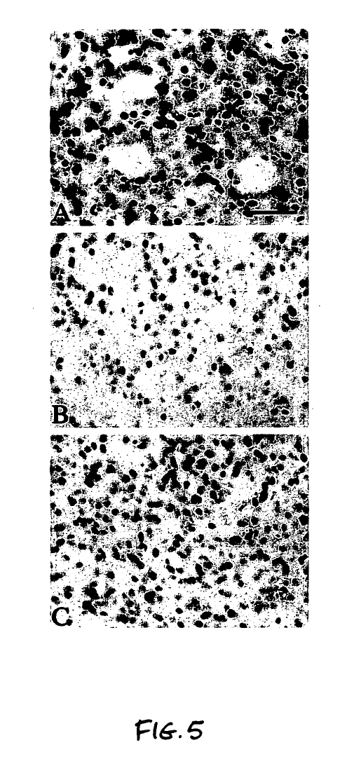

At eight weeks of age, Western blot analysis showed hypoacetylation of H3 and H4 in R6/2 mice, as compared to wild type mice, with a marked increase in H3 and H4 acetylation in sodium butyrate-treated R6/2 mice (1.2 g/kg/d) (FIG. 4). We did not observe any difference in increased acetylation of H3 and H4 between the sodium butyrate doses at 600 mg/kg/d and 1.2 g/kg/d. Immunocytochemical detection of acetylated H3 and H4 confirmed these findings. There was robust H3 and H4 acetylation immunoreactivity in brain sections of wild type mice, reduced tissue H3 and H4 immunoreactivity in R6/2 mice, and greatly enhanced H3 and H4 immunostaining in sodium butyrate-treated R6/2 mice (FIG. 5).

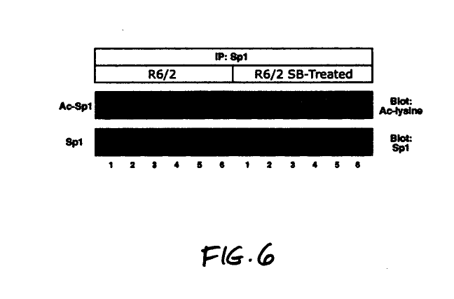

We have previously reported that Sp1 acetylation is significantly augmented in sodium butyrate-treated wild type B6CBA mice at 8 weeks of age after 2 weeks of treatment (Ryu, 2003a). Similarly, Sp1 acetylation levels were increased in brains of sodium butyrate-treated R6/2 mice (1.2 g/kg/day), as determined by immunoprecipitation using an Sp1 antibody, followed by immunoblotting using acetyl lysine antibody (Ac-Sp1) or Sp1 antibody alone. The basal levels of Sp1 did not change with sodium butyrate treatment (FIG. 6).

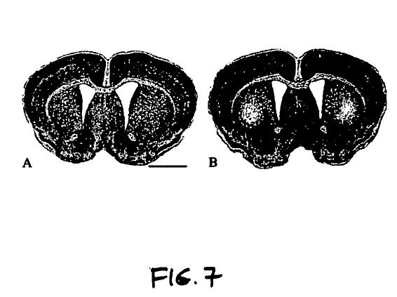

Sodium butyrate treatment for two weeks (1.2 g/kg/d), with administration of 3-NP during continuous sodium butyrate treatment started the second week, resulted in marked neuroprotection from 3-NP striatal damage in R6/2 mice, as compared to 3-NP and PBS-treated R6/2 mice (FIG. 7). Only two of ten 3-NP/sodium butyrate-treated R6/2 mice had small bilateral lesions, while seven of ten 3-NP/PBS-treated R6/2 mice had small to large bilateral striatal lesions. The histopathological evaluation of 3-NP induced striatal lesion volumes, as determined by Nissl staining, showed significantly less tissue damage in sodium butyrate-treated mice than in PBS-treated R6/2 mice (sodium butyrate-treated 3-NP R6/2 mice: 2.94 Ò 0.38 mm3; PBS-treated 3-NP R6/2 mice: 11.03 Ò 2.57 mm3; p<0.001). There was a 73.3% reduction in lesion volume in the sodium butyrate-treated R6/2 mice.

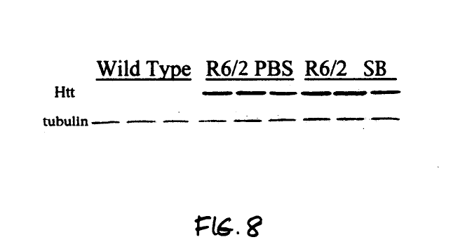

Huntingtin expression is regulated by exogenous promoters in the R6/2 transgenic mice. Treatments modulating transcription could work by suppressing the expression of the huntingtin transgene. Western analysis showed no differences in transprotein expression-levels between sodium butyrate-treated R6/2 mice and PBS-treated R6/2 mice (FIG. 8).

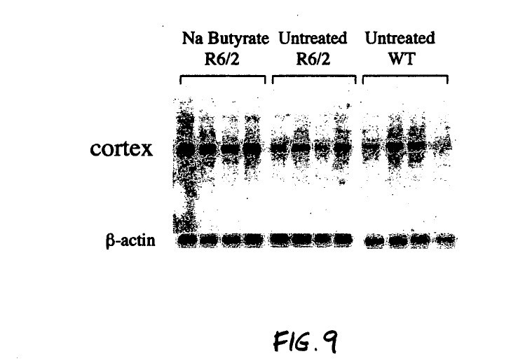

Microarray gene expression profiling was performed in striatal and cortical tissues from sodium butyrate-treated and untreated R6/2 and wild-type mice. Consistent with previous data on R6/2 mice, 69 probe sets reported changes in R6/2 striatum, as compared to wild-type striatum. These included decreases in preproenkephalin (2 probe sets), ryanodine receptor, insulin-like growth factor binding protein 5, neuronatin, T-box brain gene 1, and increases in apolipoprotein D and _{—}2 microglobulin, as previously observed. Eighteen changes in gene expression in R6/2 cerebral cortex, due to genotype alone, included decreases in complexin 11, troponin C, myosin light chain, and IGFBP5. There was a selective change in gene expression in response to sodium butyrate treatment. We observed 20 mRNA changes in sodium butyrate-treated R6/2 mice (Table 1). We focused on the most robust changes in which there were 100% possible change calls in both striatum and cortex. Among the mRNAs in R6/2 mice with these inclusion criteria, three mRNAs were detected in all eight of the array comparisons. These specific mRNAs were alpha and beta globin and MAP kinase phosphatase-1 (MKP-1) (Table 2). The increased expression of these mRNAs was confirmed using real-time PCR and Northern blotting (FIG. 9). While sodium butyrate treatment altered gene expression in R6/2 mice, some gene expression changes previously reported in R6/2 mice were not corrected. The expression of preproenkephalin mRNA, normally decreased in R6/2 mice, was unaffected by treatment. These findings represent a selective change in gene expression in response to sodium butyrate treatment and do not alter global mRNA expression. Moreover, there is not a uniform upregulation of mRNAs that are normally decreased by the disease process.

Discussion

Despite great progress, a causal pathway from the HD gene mutation to neuronal dysfunction and death has not yet been established. Early molecular events likely trigger cascades of damage and compensatory responses, leading to dysfunctional neurons that are susceptible to other insults, such as oxidative injury, excitotoxic stress, inflammatory and pro-apoptotic signals, and energy depletion (Beal, 2000; Friedlander 2003). Neuroprotective therapies targeted at specific molecular mechanisms have the potential to dramatically delay the onset and slow the progression of disease in transgenic mouse models of HD (Ferrante et al., 2000, 2002a, and 2002b; Chen et al., 2000; Andreassen et al., 2001 and 2002; Dedeoglu et. al., 2002, 2003; Karpuj et al., 2002; Hockly et al., 2003). Several pilot clinical trials in HD patients have recently been initiated from the findings observed in mouse trials. Experimental evidence increasingly points to a proximal toxicity residing in mutant huntingtin or its cleaved products and their pathological interactions with other proteins, including transcription factors (Cha 2000; Sugars and Rubinsztein 2003). Thus, therapies aimed at transcriptional modulation might target early events in HD pathogenesis and ameliorate secondary pathologic cascades.

In the present experiments, we show that intraperitoneal administration of the HDAC inhibitor, sodium butyrate, significantly extends survival in the R6/2 model of HD. Survival is a powerful and relevant endpoint for neuroprotection that provides a context for other aspects of the phenotype, such as behavior and neuropathology, and enables a ready comparison of the relative potency of different treatments. Sodium butyrate treatment also improved motor performance, gross brain weight and atrophy, and striatal neuron atrophy. While sodium butyrate treatment was neuroprotective, it did not prevent body weight loss, a finding similar to other transcriptionally active compounds (Ferrante et al., 2002b; Hockly et al., 2003). Sodium butyrate also failed to attenuate huntingtin aggregation. We previously observed this phenomenon, using the transcription modulator, mithramycin (Ferrante et al., 2002b). This suggests that neuroprotective therapies need not affect huntingtin aggregation, and supports the view that insoluble aggregates may not be inherently toxic. The specificity of neuronal vulnerability in HD may be more dependent upon soluble monomers or hetero-oligomers of huntingtin found in affected neurons (Sisodia 1998; Kuemmerle et al., 1999) and in soluble protein-protein interactions, including interactions with transcription factors. In addition, brain tissue levels of acetylated H3 and H4 activity in Western analysis and by immunocytochemistry were increased in sodium butyrate-treated R6/2 mice, consistent with a direct neuroprotective effect related to acetylation in brain. These findings demonstrate that sodium butyrate has significant efficacy in improving the neurological and neuropathological phenotype observed in the R6/2 transgenic model of HD, and suggests that HDAC inhibitors may provide clinical benefit to HD patients, most likely by preventing the deleterious effects of mutant huntingtin on transcription.

Mutant huntingtin and other polyglutamine-containing proteins can directly interact with a number of transcription factors, resulting in altered co-activation, repression, or de-repression (Cha, 2000). These include p53 and CREB-binding protein (Steffan et al., 2000), TATA-binding protein (Nakamura et al., 2001), mSin3a (Steffan et al., 2000), and Sp1 (Dunah et al., 2002; Li et al., 2002). While soluble interactions may be most toxic, many transcription factors are also directly sequestered into nuclear inclusions formed by polyglutamine-containing mutant huntingtin (Preisinger et al., 1999; McCampbell, et al., 2000; Steffan, et al., 2001; Nucifora, et al., 2001; Holbert, et al., 2001 and 2003). HDAC inhibitors selectively increase transcription due to the specificity of acetylation targets (Struhl, K, et al., 1998; Richon et al., 2000). Thus, they may only be able to reverse effects exerted through a portion of the transcriptional machinery. Sp1 is a transcription factor binding to GC rich DNA sites (Miller et al., 1991). While Sp1 has been suggested to regulate the expression of housekeeping genes, it has also been reported to be involved in differentiation, proliferation, and other cellular functions (Krainc et al., 1998; Black et al., 1999; Ryu et al., 2003a and 2003b). Polyglutamine expansion may result in neuronal death, in part, by directly affecting Sp1 (Dunah et al., 2002; Li et al., 2002). We have previously reported that HDAC inhibitors increase Sp1 acetylation and provide neuroprotection in a model of oxidative stress (Ryu et al, 2003b). The augmentation of Sp1 acetylation in R6/2 mice treated with sodium butyrate, may partially explain the observed neuroprotection.

In the present study, microarray analysis showed increased expression of MAP kinase phosphatase-1 (MKP1) and alpha and beta globins in sodium butyrate-treated R6/2 mice. The mitogen-activated protein kinase (MAPK) system is an important intracellular signaling pathway that regulates transcription and other cellular effectors, such as phospholipase C, protein kinase C, cytoplasmic phospholipase-A2, and MKP1 (Nishibe et al., 1990; Wood et al., 1992; Nemenoff et al., 1993). This regulatory network functions through phosphorylation and de-phosphorylation reactions. MKP-1 is a tyrosine phosphatase that de-phosphorylates MAPKs, thereby inactivating them. MAPKs are involved in maintaining multiple neuronal functions that are associated with HD pathogenesis, including synaptic vesicle biogenesis, glutamate release, neurite outgrowth and maintenance, and apoptosis (Brondello et al., 1999). It has been suggested that increased MKP1 concentrations alter the steady state activity of MAPK, improving cellular function without triggering entrance into the cell-cycle (Bhalla et al., 1999; 2002). Histone acetylation induces MKP1 transcription, elevating MKP1 mRNA (Li et al., 2001). Since MKP1 may regulate the transcriptional machinery, increased MPK1 may contribute to the observed neuroprotection in sodium butyrate-treated R6/2 mice. We are currently examining whether over-expression of MKP1 and ERK1 inhibition ameliorate huntingtin pathology. Increased expression of MKP1 has been reported in an in vitro model of polyglutamine-induced cell death in PC12 cells (Wu et al., 2002). While the authors suggest that the induction of MKP1 is associated with mutant huntingtin and cell death, increased MKP1 expression may be a failed attempt at cellular recovery in their model, rather than a pathological event.

Alpha and beta globins also showed increased expression in sodium butyrate-treated R6/2 mice. Enhanced globin gene expression has been reported in sodium butyrate-treated patients with thalassemia and for Hb SS (Miller, et al., 1987a; Perrine et al., 1989). There is evidence that globins are expressed in neurons and that this expression is developmentally regulated (Ohyagi et al., 1994). In addition to myoglobin and hemoglobin, a third hemoprotein, neuroglobin, has recently been isolated from vertebrate brain (Mammen et al., 2002). Overexpression of this protein protects against ischemia in an in vivo model of stroke (Sun et al., 2003). Globins contain a heme-binding domain and participate in diverse processes such as oxygen transport, oxygen storage, and nitric oxide detoxification. A secondary consequence of the HD gene defect may be impaired energy metabolism that may lead to increased free radical generation and oxidative damage (Beal, 2000; Browne et al., 1999; Tabrizi et al. 2000; Bogdanov et al., 2001). Increased globin expression may provide neuroprotective properties that enhance oxidative phosphorylation in R6/2 mice, counterbalancing HD-related metabolic deficits.

3-NP is a mitochodrial toxin that results in irreversible inhibition of succinate dehydrogenase, inhibiting both the Krebs cycle and Complex II activity of the electron transport chain. Complex II is an entrance pathway into the electron transport chain, where it reduces ubiquinone (coenzyme Q), an important electron carrier to Complex III. 3-NP produces excitotoxic-mediated striatal lesions in both man and experimental animals, closely resembling the pathology observed in HD (Alston et al., 1977; Ludolph et al., 1991; Beal et al., 1993; Brouillet et al., 1995). 3-NP reduces cellular levels of ATP and produces profound energy deficits within cells, irreversibly inhibiting the Krebs cycle and Complex II activity, resulting in an energy deficient hypoxia (Hamilton and Gould, 1987; Novelli, et al., 1988; Ludolph et al., 1992). Sodium butyrate enhances Sp1 acetylation and inhibits 3-NP-induced excitotoxicity in vivo (Ryu et al., 2003a and 2003b). In the current experiments, increased acetylation of Sp1 induced by sodium butyrate treatment was associated with nearly complete protection from striatal 3-NP toxicity. Resistance to 3-NP toxicity in sodium butyrate-treated R6/2 mice may be due, in part, to improved oxidative metabolism via increased globin expression.

We show that sodium butyrate is neuroprotective in R6/2 transgenic mice. There are a number of possible explanations for this neuroprotection and it is likely that more than one contributes. Sodium butyrate acts at the transcription level by increasing the acetylation of histones, thereby, releasing constraints on the DNA template and reactivating a number of genes (Sealy et al., 1978; Candido et al., 1978). Using the percentage increase in survival as a standard, sodium butyrate is among the most efficacious compounds yet tested. Since the butyrates have known oral and CNS bioavailability and since toxicity has been low and tolerability acceptable in both human and animal studies (Collins et al.,1995; Egorin et al., 1999), sodium butyrate and related compounds are promising neuroprotective agents for HD that warrant further preclinical testing and possible consideration for clinical trials in the future. An important implication of the multiple levels of molecular pathology existing in HD is that it will most likely be possible to combine neuroprotective therapies to maximize efficacy. HDAC inhibitors, including the butyrates, may act more proximally and are candidates for combination with other neuroprotective compounds currently being developed for HD.

TABLE 1
Table 1: Changes in gene expression in sodium butyrate-treated R6/2 mice.

| | | | | Direction | % |
|---|---|---|---|---|---|
| | | | | of | Change |
| Brain region | Genbank ID | Probe ID | mRNA | change | calls |
| Striatum + Cortex | X61940 | 104598_at | MAP kinase phosphatase-1 (MKP-1) | Increase | 100 |
| Striatum + Cortex | V00714 | 94781_at | Hemoglobin, alpha adult chain 1 | Increase | 100 |
| Striatum + Cortex | J00413 | 101869_s_at | Hemoglobin, beta adult major chain | Increase | 100 |
| Cortex | V00722 | 103534_at | Hemoglobin, beta 1 | Increase | 75 |
| Cortex | V00727 | 160901_at | c-fos oncogene | Increase | 100 |
| Cortex | AI854404 | 160131_at | Angiomotin like 2 | Increase | 100 |
| Cortex | AI845584 | 93285_at | MAP kinase phosphatase-3 (MKP-3) | Increase | 75 |
| Cortex | M88354 | 93381_at | Arginine vasopressin | Increase | 75 |
| Striatum | D45859 | 101836_at | Protein phosphatase 1B, beta isoform | Increase | 75 |
| Striatum | AB028272 | 96254_at | DNAJ/Heat shock protein 40 | Increase | 75 |
| Striatum + Cortex | C79248 | 94689_at | Unknown | Decrease | 75 |
| Cortex | L31397 | 103031_g_at | Dynamin | Decrease | 75 |
| Cortex | M18775 | 102431_at | Microtubule-associated protein tau | Decrease | 75 |
| Cortex | AV330064 | 161114_i_at | Unknown | Decrease | 75 |
| Striatum | AJ007909 | 98525_f_at | Erythroid differentiation regulator | Decrease | 100 |
| Striatum | AI451558 | 97161_at | Unknown | Decrease | 75 |
| Striatum | AI153421 | 96215_f_at | Unknown | Decrease | 75 |
| Striatum | D00073 | 95350_at | Transthyretin | Decrease | 75 |
| Striatum | AA711516 | 96302_at | Unknown | Decrease | 75 |
| Striatum | AI183202 | 92724_at | Heterogeneous nuclear ribonucleoprotein A1 | Decrease | 75 |

Samples from sodium butyrate-treated R6/2, untreated R6/2, and wild-type mice (n=4 from each group) were analyzed for differential mRNA expression by microarray. Array data are expressed as increased or decreased based upon a minimum Difference Call cutoff of three of four independent comparisons using samples from individual mice (see Methods). Most important in the gene array findings was that three known genes, alpha and beta globin and MAP kinase phosphatase-1, were highly increased in expression. These increases in mRNAs were specific to both the striatum and neocortex and were detected in all eight array comparisons.

TABLE 2
Table 2: Changes in alpha and beta globin and MAP kinase phosphatase-1
gene expression in sodium butyrate-treated R6/2 mice.

| | | NaBu Txd R6/2 vs R6/2 | | | Untreated R6/2 vs WT | | |
|---|---|---|---|---|---|---|---|
| mRNA | Region | Array | PCR | Northern | Array | PCR | Northern |
| MKP-1 | Striatum | I | 181%^{a} | | NC | 75% | |
| | Cortex | I | | 204%^{e} | NC | | 81% |
| α-globin | Striatum | I | 206%^{c} | | NC | 120% | |
| ε | Cortex | I | | 159%^{c} | NC | | 99% |
| β-globin | Striatum | I | 215%^{b} | | NC | 104% | |
| ε | Cortex | I | | 206%^{d} | D | | 39%^{a} |
| Preproenkephalin | Striatum | NC | 101% | 98% | D | 52%^{b} | 62%^{a} |
| Samples from sodium butyrate-treated R6/2, untreated R6/2, and wild-type mice (n = 4 from each group) were analyzed for differential mRNA expression by microarray, real-time PCR and northern blotting. Array data are expressed as I = increased, D = decreased, or NC = no change, based on a minimum Difference Call cutoff of three of four independent comparisons using | | | | | | | |
| # samples from individual mice (see Methods). PCR and northern data are presented as mean values expressed as a percentage of the baseline group (either untreated R6/2 or wild-type mice) after normalization to β-actin signal (see Methods). | | | | | | | |

^{a}Significantly different from comparison group p < 0.05;
^{b}Significantly different from comparison group p < 0.02;
^{c}Significantly different from comparison group p < 0.01;
^{d}Significantly different from comparison group p < 0.001;
^{e}Significantly different from comparison group p < 0.0001.

Samples from sodium butyrate-treated R6/2, untreated R6/2, and wild-type mice (n=4 from each group) were analyzed for differential mRNA expression by microarray, realtime PCR and northern blotting. Array data are expressed as I=increased, D=decreased, or NC=no change, based on a minimum Difference Call cutoff of three of four independent comparisons using samples from individual mice (see Methods). PCR and northern data are presented as mean values expressed as a percentage of the baseline group (either untreated R6/2 or wild-type mice) after normalization to β-actin signal (see Methods). a Significantly different from comparison group p<0.05; b Significantly different from comparison group p<0.02; c Significantly different from comparison group p<0.01; d Significantly different from comparison group p<0.001; e Significantly different from comparison group p<0.0001.

While this invention has been described as having preferred sequences, ranges, steps, materials, structures, features, and/or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the appended claims.

### REFERENCES

The following references, and those cited in the disclosure herein, are hereby incorporated herein in their entirety by reference.
1. Alston, T A, Mela L, Bright H J (1977) 3-Nitropropionate, the toxic substance of Indigofera, is a suicide inactivator of succinate dehydrogenase. Proc Natl Acad Sci 74: 3767-3771.
2. Andreassen O A, Ferrante R J, Huang H M, Dedeoglu A, Park L, Ferrante K L, Kwon J, Borchelt D R, Ross C A, Gibson G E, Beal M F (2001) Dichloroacetate exerts therapeutic effects in transgenic mouse models of Huntington's disease. Ann Neurol 50: 112-117.
3. Andreassen O A, Ferrante R J, Dedeoglu A, Beal M F (2001) Lipoic acid improves survival in transgenic mouse models of Huntington's disease. Neuroreport 12:3371-3374.
4. Beal M F (2000) Energetics in the pathogenesis of neurodegenerative diseases. Trends Neurosci 7:298-304.
5. Beal M F, Brouillet E, Jenkins B G, Ferrante R J, Kowall N W, Miller J M, Storey E, Srivastava R, Rosen B R, Hyman B T (1993) Neurochemical and histologic characterization of striatal excitotoxic lesions produced by the mitochondrial toxin 3-nitropropionic acid. J Neurosci 13: 4181-4192.
6. Bhalla, U S, Ram P T, Iyengar R (2002) MAP kinase phosphatase as a locus of flexibility in a mitogen-activated protein kinase signaling network. Science 297:1018-1023.
7. Bhalla U S, lyengar R (1999) Emergent properties of networks of biological signaling pathways. Science 283:381-387.
8. Bogdanov M B, Andreassen O A, Dedeoglu A, Ferrante R J, Beal M F (2001) Increased oxidative damage to DNA in a transgenic mouse model of Huntington's disease. J Neurochem 79:1246-1249.
9. Browne S E, Ferrante R J, Beal M F (1999) Oxidative stress in Huntington's disease. Brain Pathol 9:147-163.
10. Brouillet E, Hantraye P, Ferrante R J, Dolan R, Kowall N W, Beal M F (1995) Chronic mitochondrial energy impairment produces selective striatal degeneration and abnormal choreiform movements in primates. Proc Natl Acad Sci USA, 92:7105-7109.
11. Black A R, Jensen D, Lin S Y, Azizkhan J C (1999) Growth/cell cycle regulation of Sp1 phosphorylation. J Biol Chem 274:1207-1215.
12. Brondello J M, Pouyssegur J, McKenzie F R (1999) Reduced MAP kinase phosphatase-1 degradation after p42/p44MAPK-dependent phosphorylation. Science. 286:2514-2517.
13. Butler L M, Agus D B, Scher H I, Higgins B, Rose A, Cordon-Cardo C, Thaler H T, Rifkind R A, Marks P A, Richon V M (2000) Suberoylanilide hydroxamic acid, an inhibitor of histone deacetylase, suppresses the growth of prostate cancer cells in vitro and in vivo. Cancer Res 60:5165-5170.
14. Candido E P, Reeves R, Davie J R (1978) Sodium butyrate inhibits histone deacetylation in cultured cells. Cell. 14:105-113.
15. Cha J H (2000) Transcriptional dysregulation in Huntington's disease. Trends Neurosci 23:387-392.
16. Chatterjee S, Zaman K, Ryu H, Conforto A, Ratan R R (2001) Sequence-selective DNA binding drugs mithramycin A and chromomycin A3 are potent inhibitors of neuronal apoptosis induced by oxidative stress and DNA damage in cortical neurons. Ann Neurol 49:345-354.
17. Chen M, Ona V O, Li M, Ferrante R J, Fink K B, Zhu S, Bian J, Guo L, Farrell L A, Hersch S M, Hobbs W, Vonsattel J P, Cha J H, Friedlander R M (2000) Minocycline inhibits caspase-1 and caspase-3 expression and delays mortality in a transgenic mouse model of Huntington disease. Nat Med 6: 797-801.
18. Collins A F, Pearson H A, Giardina P, McDonagh K T, Brusilow S W, Dover G J (1995) Oral sodium phenylbutyrate therapy in homozygous beta thalassemia: a clinical trial. Blood 85:43-49.
19. Dedeoglu A, Kubilus J K, Jeitner T M, Matson S A, Bogdanov M, Kowall N W, Matson W R, Cooper A J, Ratan R R, Beal M F, Hersch S M, Ferrante R J (2002) Therapeutic effects of cystamine in a murine model of Huntington's disease. J Neurosci 22:8942-8950.
20. Dedeoglu A, Kubilus J K, Yang L, Ferrante K L, Hersch S M, Beal M F, Ferrante R J (2003) Creatine Therapy Provides Neuroprotection After Onset of Clinical Symptoms in Huntington's Disease Transgenic Mice. J Neurochem 85: 1359-1367.
21. Dunah A W, Jeong H, Griffin A, Kim Y M, Standaert D G, Hersch S M, Mouradian M M, Young A B, Tanese N, Krainc D (2002) Sp1 and TAFII130 Transcriptional Activity Disrupted in Early Huntington's Disease. Science 296:2238-2243.
22. Egorin M J, Yuan Z M, Sentz D L, Plaisance K, Eiseman J L (1999) Plasma pharmacokinetics of butyrate after intravenous administration of sodium butyrate or oral administration of tributyrin or sodium butyrate to mice and rats. Cancer Chemother Pharmacol 43:445-453.
23. Ferrante R J, Andreassen O A, Jenkins B G, Dedeoglu A, Kuemmerie S, Kubilus J K, Kaddurah-Daouk R, Hersch S M, Beal M F (2000) Neuroprotective effects of creatine in a transgenic mouse model of Huntington's disease. J Neurosci 20:4389-97.
24. Ferrante R J, Andreassen O A, Dedeoglu A, Ferrante K L, Jenkins B G, Hersch S M, Beal M F (2002a) Therapeutic effects of coenzyme Q10 and remacemide in transgenic mouse models of Huntington's disease. J Neurosci 22:1592-1599.
25. Ferrante R J, Dedeoglu A, Kubilus J K, Sugars K L, Rubinsztein D C, Ryu H, Lee J H, Beal M F, Ratan R R (2002b) Therapeutic effects of mithramycin in R6/2 transgenic Huntington's disease mice. SFN Abstr.
26. Ferrante R J, Kubilus J K, Lee J, Ryu H, Beesen A, Zucker B, Smith K, Kowall N W, Ratan R R, Luthi-Carter R, Hersch S M (2003) Histone Deacetylase Inhibition by Sodium Butyrate Chemotherapy Ameliorates the Neurodegenerative Phenotype in Huntington's Disease Mice. J Neurosci 23(28):9418-9427.
27. Friedlander R M (2003) Apoptosis and caspases in neurodegenerative diseases. N Engl J Med 348:1365-1375.
28. Hamilton B F, Gould D H (1987) Nature and distribution of brain lesions in rats intoxicated with 3-nitropropionic acid: a type of hypoxic (energy deficient) brain damage. Acta Neuropathol 72: 286-297.
29. Hockly E, Richon V M, Woodman B, Smith D L, Zhou X, Rosa E, Sathasivam K, Ghazi-Noori S, Mahal A, Lowden P A, Steffan J S, Marsh J L, Thompson L M, Lewis C M, Marks P A, Bates G P (2003) Suberoylanilide hydroxamic acid, a histone deacetylase inhibitor, ameliorates motor deficits in a mouse model of Huntington's disease. Proc Natl Acad Sci USA 100:2041-2046.
30. Holbert S, Denghien I, Kiechle T, Rosenblatt A, Wellington C, Hayden M R, Margolis R L, Ross C A, Dausset J, Ferrante R J, Neri C (2001) The Gin-Ala repeat transcriptional activator CA150 interacts with huntingtin: neuropathologic and genetic evidence for a role in Huntington's disease pathogenesis. Proc Natl Acad Sci USA 98:1811-1816.
31. Holbert S, Dedeoglu A, Humbert S, Saudou F, Ferrante R J, Neri C (2003) Cdc42-interacting protein 4 binds to huntingtin: neuropathologic and biological evidence for a role in Huntington's disease. Proc Natl Acad Sci USA 100:2712-2717.
32. Karpuj M V, Becher M W, Springer J E, Chabas D, Youssef S, Pedotti R. Mitchell D, Steinman L (2002) Prolonged survival and decreased abnormal movements in transgenic model of Huntington disease, with administration of the transglutaminase inhibitor cystamine. Nat Med 8:143-149.
33. Krainc D, Bai G, Okamoto S, Carles M, Kusiak J W, Brent R N, Lipton S A (1998) Synergistic activation of the N-methyl-D-aspartate receptor subunit 1 promoter by myocyte enhancer factor 2C and Sp1. J Biol Chem 273:26218-26224.
34. Kuemmerle S, Gutekunst C A, Klein A M, Li X J, Li S H, Beal M F, Hersch S M, Ferrante R J (1999) Huntington aggregates may not predict neuronal death in Huntington's disease. Ann Neurol 46: 842-849.
35. Li J, Gorospe M, Hutter D, Barnes J, Keyse S M, Liu Y (2001) Transcriptional induction of MKP-1 in response to stress is associated with histone H3 phosphorylation-acetylation. Mol Cell Biol. 23:8213-8224.
36. Li S H, Cheng A L, Zhou H, Lam S, Rao M, Li H, Li X J (2002) Interaction of Huntington disease protein with transcriptional activator Sp1. Mol Cell Biol 22:1277-1287.
37. Livak K J and Schmittgen T D (2001) Analysis of relative gene expression data using real-time quantitative PCR and the 2(-Delta Delta C(T)) Methods 25:402-408.
38. Ludolph A C, He F, Spencer P S (1991) 3-Nitropropionic acid-exogenous animal neurotoxin and possible human striatal toxin. Can J Neurol Sci 18:492-498.
39. Ludolph, A C, Seelig M, Ludolph A (1992) 3-Nitropropionic acid decreases cellular energy levels and causes neuronal degeneration in cortical explants. Neurodegeneration 1:155-161.
40. Luthi-Carter R, Strand A, Peters N L, Solano S M, Hollingsworth Z R, Menon A S, Frey A S, Spektor B S, Penney E B, Schilling G, Ross C A, Borchelt D R, Tapscott S J, Young A B, Cha J H, Olson J M (2000) Decreased expression of striatal signaling genes in a mouse model of Huntington's disease. Hum Mol Genet 9:1259-1271.
41. Luthi-Carter R, Hanson S A, Strand A D, Bergstrom D A, Chun W, Peters N L, Woods A M, Chan E Y, Kooperberg C, Krainc D, Young A B, Tapscott S J, Olson J M (2002) Dysregulation of gene expression in the R6/2 model of polyglutamine disease: parallel changes in muscle and brain. Hum Mol Genet 11:1911-1926.
42. Mammen P P, Shelton J M, Goetsch S C, Williams S C, Richardson J A, Garry M G, Garry D J (2002) Neuroglobin, a novel member of the globin family, is expressed in focal regions of the brain. J Histochem Cytochem 50:1591-1598.
43. McCampbell A, Taylor J P, Taye A A, Robitschek J, Li M, Walcott J, Merry D, Chai Y, Paulson H, Sobue G, Fischbeck K H (2000) CREB-binding protein sequestration by expanded polyglutamine. Hum Mol Genet 9: p. 2197-2202.
44. McCampbell A, Taye A A, Whitty L, Penney E, Steffan J S, Fischbeck K H (2001) Histone deacetylase inhibitors reduce polyglutamine toxicity. Proc Natl Acad Sci USA 98:15179-15184.
45. Miller A A, Kurschel E, Osieka R, Schmidt C G (1987a) Clinical pharmacology of sodium butyrate in patients with acute leukemia. Eur J Cancer Clin Oncol 23:1283-1287.
46. Miller D M, Polansky D A, Thomas S D, Ray R, Campbell V W, Sanchez J, Koller C A (1987b) Mithramycin selectively inhibits transcription of G-C containing DNA. Am J Med Sci 294:388-394.
47. Nakamura K, Jeong S Y, Uchihara T, Anno M, Nagashima K, Nagashima T, Ikeda S, Tsuji S, Kanazawa I (2001) SCA17, a novel autosomal dominant cerebellar ataxia caused by an expanded polyglutamine in TATA-binding protein. Hum Mol Genet 10:1441-1448.
48. Nemenoff R A, Winitz S, Qian N X, Van Putten V, Johnson G L, Heasley L E (1993) Phosphorylation and activation of a high molecular weight form of phospholipase A2 by p42 microtubule-associated protein 2 kinase and protein kinase C. J Biol Chem 268:1960-1964.
49. Nishibe S, Wahl M I, Hernandez-Sotomayor S M, Tonks N K, Rhee S G, Carpenter G (1990) Increase of the catalytic activity of phospholipase C-gamma 1 by tyrosine phosphorylation. Science 250:1253-1256.
50. Novelli A, Reilly J A, Lysko P G, Henneberry R C (1988) Glutamate becomes neurotoxic via the N-methyl-D-aspartate receptor when intracellular energy levels are reduced. Brain Res 451:205-212.
51. Nucifora F C, Jr, Sasaki M, Peters M F, Huang H, Cooper J K, Yamada M, Takahashi H, Tsuji S, Troncoso J, Dawson V L, Dawson T M, Ross C A (2001) Interference by huntingtin and atrophin-1 with cbp-mediated transcription leading to cellular toxicity. Science 291: 2423-2428.
52. Ohyagi Y, Yamada T, Goto I (1994) Hemoglobin as a novel protein developmentally regulated in neurons. Brain Res 635:323-327.
53. Perrine S P, Miller B A, Faller D V, Cohen R A, Vichinsky E P, Hurst D, Lubin B H, Papayannopoulou T (1989) Sodium butyrate enhances fetal globin gene expression in erythroid progenitors of patients with Hb SS and beta thalassemia. Blood 74:454-459.
54. Preisinger E, Jordan B M, Kazantsev A, Housman D (1999) Evidence for a recruitment and sequestration mechanism in Huntington's disease. Philos Trans R Soc Lond B Biol Sci 354: 1029-1034.
55. Richon V M, Sandhoff T W, Rifkind R A, Marks P A (2000) Histone deacetylase inhibitor selectively induces p21WAF1 expression and gene-associated histone acetylation. Proc Natl Acad Sci 97:10014-10019.
56. Ryu H, Lee J, Olofsson B A, Mwidau A, Deodoglu A, Escudero M, Flemington E, Azizkhan-Clifford J, Ferrante R J, Ratan R R (2003a) Histone deacetylase inhibitors prevent oxidative neuronal death independent of expanded polyglutamine repeats via an Sp1-dependent pathway. Proc Natl Acad Sci USA 100:4281-4286.
57. Ryu H, Lee J H, Zaman K, Ferrante R J, Ross B D, Neve R, Ratan R R (2003b) Sp1 and Sp3 are oxidative stress-inducible, anti-death transcription factors in cortical neurons. J. Neurosci 23:3597-3606.
58. Sealy L and Chalkley R (1978) The effect of sodium butyrate on histone modification. Cell 14:115-121.
59. Sisodia S S (1998) Nuclear inclusions in glutamine repeat disorders: Are they pernicious, coincidental, or beneficial? Cell 95: 1-4.
60. Steffan J S, Kazantsev A, Spasic-Boskovic O, Greenwald M, Zhu Y Z, Gohler H, Wanker E E, Bates G P, Housman D E, Thompson L M (2000) The Huntington's disease protein interacts with p53 and CREB-binding protein and represses transcription. Proc Natl Acad Sci USA 97:6763-6768.
61. Steffan J S, Bodai L, Pallos J, Poelman M, McCampbell A, Apostol B L, Kazantsev A, Schmidt E, Zhu Y Z, Greenwald M, Kurokawa R, Housman D E, Jackson G R, Marsh J L, Thompson L M (2001) Histone deacetylase inhibitors arrest polyglutamine-dependent neurodegeneration in Drosophila. Nature 413: 739-743.
62. Struhl K (1998) Histone acetylation and transcriptional regulatory mechanisms. Genes Dev 12:599-606.
63. Sugars K L and Rubinsztein D C (2003) Transcriptional abnormalities in Huntington disease. TRENDS in Genetics 19: 233-238.
64. Sun Y, Jin K, Peel A, Mao X O, Xie L, Greenberg D A (2003) Neuroglobin protects the brain from experimental stroke in vivo. Proc Natl Acad Sci USA 100: 3497-3500.
65. Tabrizi S J, Workman J, Hart P E, et al. (2000) Mitochondrial dysfunction and free radical damage in the Huntington R6/2 transgenic mouse. Ann Neurol 47: 80-86.
66. Warrell R P, Jr., He, L Z, Richon V, Calleja E, Pandolfi P P (1998) Therapeutic targeting of transcription in acute promyelocytic leukemia by use of an inhibitor of histone deacetylase. J Natl Cancer Inst 90: 1621-1625.
67. Wood K W, Sarnecki C, Roberts T M, Blenis J (1992) Ras mediates nerve growth factor receptor modulation of three signal-transducing protein kinases: MAP kinase, Raf-1, and RSK.Cell 68:1041-1050.
68. Wu Z L, O'Kane T M, Scott R W, Savage M J, Bozyczko-Coyne D (2002) Protein tyrosine phosphatases are up-regulated and participate in cell death induced by polyglutamine expansion. J Biol Chem 77:44208-44213.
69. Van Lint C, Emiliani S, Verdin E (1996) The expression of a small fraction of cellular genes is changed in response to histone hyperacetylation. Gene Expr 5:245-253.
70. Vigushin D M, Coombes R C (2002) Histone deacetylase inhibitors in cancer treatment. Anticancer Drugs 13:1-13.

What is claimed is:

1. A method of ameliorating or abrogating the effects of a neurodegenerative disorder in a subject, comprising:
administering a histone decaetylase (HDAC) inhibiting agent in a subject in need thereof;
wherein the HDAC inhibiting agent comprises a butyrate.

2. The method of claim 1, wherein:
the butyrate comprises sodium butyrate.

3. The method of claim 2, wherein:
the neurodegenerative disorder comprises Huntington's disease (HD).

4. A method of preventing neuronal death in a subject having Huntington's disease, comprising:
administering a histone deacetylase (HDAC) inhibiting agent in a subject in need thereof;
wherein the HDAC inhibiting agent comprises a butyrate.

5. The method of claim 4, wherein:
the butyrate comprises sodium butyrate.

6. The method of claim 4, wherein:
the HDAC inhibiting agent is administered intraperitoneally.

7. A method of treating a subject having Huntington's disease, comprising:
administering a histone deacetylase (HDAC) inhibiting agent in a subject in need thereof;
wherein the HDAC inhibiting agent comprises a butyrate.

8. The method of claim 7, wherein:
the butyrate comprises sodium butyrate.

9. The method of claim 7, wherein:
the HDAC inhibiting agent is administered intraperitoneally.

10. A method of protecting neural cells in a subject having a neurological disorder, comprising:
increasing histone acetylation by administering a butyrate in a subject in need thereof.

11. The method of claim 10, wherein:
the butyrate comprises sodium butyrate.

12. The method of claim 10, wherein:
the neurological disorder comprises Huntington's disease.

13. The method of claim 1, wherein:
the subject comprises a mammal.

14. The method of claim 1, wherein:
the subject comprises a Huntington's disease mouse.

15. A method of improving motor performance, gross brain weight and atrophy, striatal neuron atrophy, and/or survival in a subject, comprising:
administering a histone decaetylase (HDAC) inhibiting agent in a subject in need thereof;
wherein the HDAC inhibiting agent comprises a butyrate.

16. The method of claim 15, wherein:
the subject comprises a mammal.

17. The method of claim 15, wherein:
the subject comprises a Huntington's disease mouse.

18. The method of claim 17, wherein:
the survival is improved by administering a dose of the HDAC inhibiting agent ranging from about 200 mg/kg to 1200 mg/kg.


SEQUENCE LISTING (10 sequences)

SEQ ID NO: 1 - DNA, 18 bp, Unknown. Obtained from Commercial Source.
  misc_feature: Synthesized
gtgtccaggcccgagttc

SEQ ID NO: 2 - DNA, 20 bp, Unknown. Obtained from Commercial Source.
  misc_feature: Synthesized
tctcccgttcccagtagctc

SEQ ID NO: 3 - DNA, 20 bp, Unknown. Obtained from Commercial Source.
  misc_feature: Synthesized
aagcagaggcggagtatcat

SEQ ID NO: 4 - DNA, 20 bp, Unknown. Obtained from Commercial Source.
  misc_feature: Synthesized
tagttcagggcactgttcgt

SEQ ID NO: 5 - DNA, 17 bp, Unknown. Obtained from Commercial Source.
  misc_feature: Synthesized
ccaccctgccgatttca

SEQ ID NO: 6 - DNA, 19 bp, Unknown. Obtained from Commercial Source.
  misc_feature: Synthesized
ccgcagaaggcagcttaac

SEQ ID NO: 7 - DNA, 20 bp, Unknown. Obtained from Commercial Source.
  misc_feature: Synthesized
cttggacccagcggtacttt

SEQ ID NO: 8 - DNA, 19 bp, Unknown. Obtained from Commercial Source.
  misc_feature: Synthesized
cccagcacaatcacgatca

SEQ ID NO: 9 - DNA, 19 bp, Unknown. Obtained from Commercial Source.
  misc_feature: Synthesized
aggtatcctgaccctgaag

SEQ ID NO: 10 - DNA, 20 bp, Unknown. Obtained from Commercial Source.
  misc_feature: Synthesized
gctcattgtagaaggtgtgg